(19) United States
(12) Patent Application Publication
(10) Pub. No.: US 2021/0093920 A1
(43) Pub. Date: Apr. 1, 2021

(54) Personal Fitness Training System With Biomechanical Feedback

(71) Applicant: True Adherence, Inc., Aurora, CO (US)

(72) Inventors: Kyle Poulin, Salt Lake City, UT (US); Chris Bright, Provo, UT (US)

(73) Assignee: True Adherence, Inc., Aurora, CO (US)

(21) Appl. No.: 17/004,780

(22) Filed: Aug. 27, 2020

Related U.S. Application Data
(60) Provisional application No. 62/906,423, filed on Sep. 26, 2019.

(51) Int. Cl. A63B 24/00; A63B 71/06

(52) U.S. Cl. CPC A63B 24/0075; A63B 24/0062; A63B 2071/0625; A63B 2071/065; A63B 2071/0647; A63B 71/0622

(57) ABSTRACT

Techniques and apparatuses describe an example personal fitness training computing system with biomechanical feedback to provide customized personal fitness training. The computing system generates a portrayal representative of a person identified for participating in the personal fitness training. The portrayal is displayed on a display device communicating with the computing system. A physical activity of the person is monitored during the personal fitness training. Feedback relative to the physical activity is then displayed on the display device. The feedback includes a biomechanical indication of the physical activity represented with respect to the portrayal of the person.

### PRIORITY APPLICATION

This application claims priority under 35 U.S.C. § 119(e) to U.S. Provisional Patent Application No. 62/906,423, filed Sep. 26, 2019, the disclosure of which is incorporated in its entirety by reference herein.

### BACKGROUND

It is important for athletes and individuals to exercise and train properly, not only to maintain and improve their physical well-being but to avoid over-stressing or injuring specific muscles, joints, or other parts of the body being worked. As such, it is common to retain personal human trainers to assist and guide in a personal workout. But personal trainers are not always available, and may not have all relevant information available that may be helpful or even essential for a more fully customized personal fitness, training, or conditioning exercise workout program.

### BRIEF DESCRIPTION OF THE DRAWINGS

Examples of a personal fitness training system with biomechanical feedback will now be described with reference to the accompanying drawings.

FIG. 1 is a flow diagram illustrating an example method of customizing personal fitness training with biomechanical feedback.

FIG. 2 illustrates an example method of customizing personal fitness training with biomechanical feedback from the perspective of example computing devices interacting in a personal fitness training computing system.

FIG. 3 illustrates an example overview of data flow associated with a personal fitness training computing system with biomechanical feedback.

FIG. 4 illustrates an example personal fitness manager interactive display with biomechanical feedback.

FIG. 5 illustrates an example personal fitness manager interactive display with biomechanical feedback placed in front of, and being used by, a user performing a workout.

FIG. 6 illustrates example user interface features and functionality of a personal fitness manager interactive display with biomechanical feedback.

FIG. 7 depicts a personal fitness manager interactive display user-interface illustrating an example of the user's portrayal positioned side-by-side with a trainer's portrayal.

FIG. 8 illustrates an example personal fitness manager interactive display depicting user-interface features of a workout summary.

FIG. 9 is a block diagram illustrating an example computing device having a personal fitness manager interactive display.

### DETAILED DESCRIPTION

This document describes example techniques and apparatuses for customizing personal fitness training. A computing system generates a portrayal of a person identified for participating in the personal fitness training. The portrayal is displayed on a display device communicating with the computing system. A physical activity of the person is monitored during the personal fitness training. Feedback relative to the physical activity is then displayed on the display device. The feedback includes a biomechanical indication of the physical activity represented with respect to the portrayal of the person.

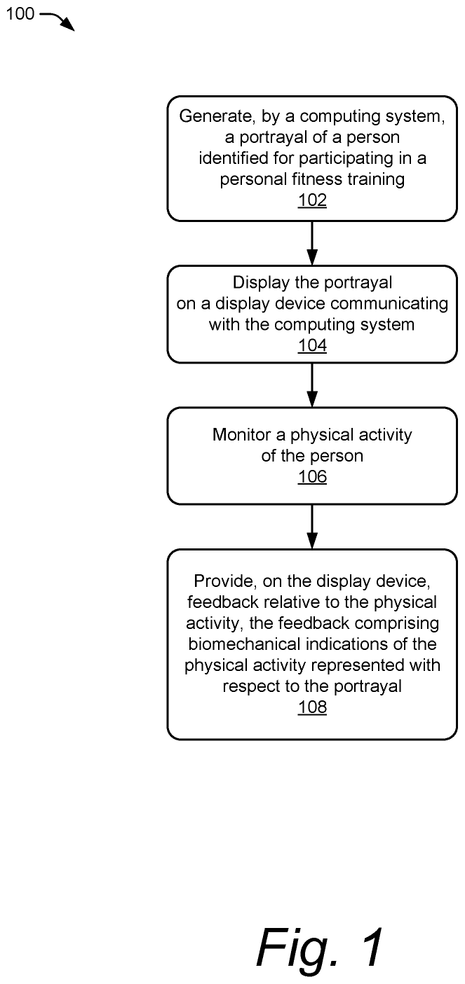

FIG. 1 is a flow diagram illustrating an example method 100 of customizing personal fitness training with biomechanical feedback. At 102, a computing system generates a portrayal of a person identified for participating in the personal fitness training. For simplicity of discussion, the term fitness training includes exercising, working out, conditioning, physical therapy movements, and similar physical movements and/or activities of a person. The computing system may include a computing device and/or separate components, for example, a computer, a monitor, a mobile device, a camera, and/or a network connection to a storage device or for computer processing, or internet connection to cloud computing and/or storage resources. The computing system may also include components combined together, such as a computer, camera, and touch screen display in the form of a single all-in-one, kiosk-type of configuration that may communicate with a network or the Internet, or may include modular components with a combined similar functionality.

The portrayal of the person may be represented in any of a number of configurations, for example as a skeletal figure, a stick figure, a caricature representation, a bodily form representation, or any other feasible representation of the person. The portrayal represents, in a sense, a representation or reflection of the person, which may include physical measurements, sufficient to allow the person to identify physical movement, joint movement, body form, alignment, positioning, and/or stress relative to the portrayal during fitness training. In one example, the portrayal may be generated using an infrared camera, or light detection and ranging camera (also referred to as a laser imaging, detection, and ranging camera, or lidar camera), or the like. The camera generates a dot map representation of the person, and then the computing system and/or associated software creates the portrayal from the dot map representation.

At 104, the portrayal is displayed on a display device (e.g., a touch screen monitor, a computer monitor, a mobile device screen) communicating with the computing system. At 106, a physical activity of the person is monitored in real time, for example, during the fitness training. The monitoring may occur using the infrared or lidar camera, or a video camera with frame-by-frame recording analysis capability. At 108, feedback relative to the physical activity is provided on the display device. The feedback includes a biomechanical indication of the physical activity of the person as represented with respect to the portrayal. For example, using skeletal points (e.g., bodily joints) associated with the portrayal, and scientifically referenced best movement patterns (e.g., kinesiology studies), the computing system provides feedback to the user, in the form of the portrayal, visual images, video, text, and/or audio comments, describing how to correct their positioning and form to do the current fitness training correctly.

This real-time feedback allows the person to quickly identify errors relative to the physical activity being performed, including as conveyed by the feedback comprising the biomechanical indication of the physical activity. The feedback may also help identify how to improve the physical activity, including as conveyed relative to the feedback comprising the biomechanical indication. For example, joint movement, body form, alignment, and/or positioning, may be detected with respect to the physical activity as represented with respect to the portrayal, and/or identified as a cue or variation displayed by the portrayal to be corrected or improved. Feedback may include visual or audio indicators. For example, the computing system may display video, an image, a color, a graphic, a line, a circle, a geometric figure, a symbol, or a written language indicator, that highlights, complements, references or supports the biomechanical indication associated with the portrayal. Feedback may also include other visual or audio indicators separate from the biomechanical feedback of the portrayal relevant to the personal fitness training. Each of these indicators may assist the person in performing and improving their customized personal fitness training.

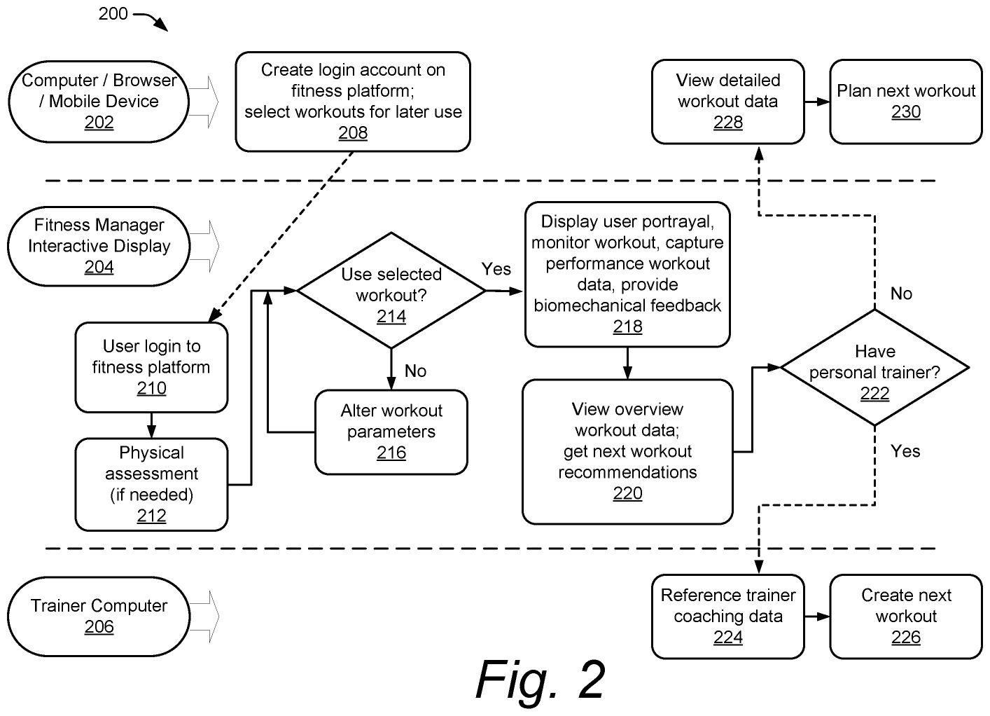

FIG. 2 illustrates an example method 200 of customizing personal fitness training with biomechanical feedback from the perspective of example computing devices interacting with a personal fitness manager interactive display device communicating in a computing system. In this example, the computing system may include, at 202, a computer, browser, or mobile device, and at 204, a fitness manager interactive display, and at 206 a fitness trainer computer. At 208, a person (e.g., user) that intends to participate in a customized personal fitness training activity, interacts with the computer, browser, or mobile device (at 202) to create a personal account, input personal data to create a personal profile, and initially select specific workouts or other activities for fitness training purposes. In an alternate embodiment, the person may create the personal account and input personal data directly into the fitness manager interactive display at 204. The personal account and personal data is stored in a central location fitness platform, for example, associated with the fitness manager interactive display and/or in the cloud or on a network server, for access by the computer, browser, or mobile device at 202, fitness manager interactive display at 204, and/or the trainer computer at 206. Dashed directional lines in the figure represent flow transition from one computing device to another computing device. Solid directional lines represent flow transition from one function of an identified computing device to another function of that same computing device.

When the person is ready to perform their fitness training, they interact with data stored on the fitness platform using, for example, the fitness manager interactive display at 204. The fitness manager interactive display may be an all-in-one type of kiosk, having a computer, camera, and interactive display. Alternatively, the fitness manager interactive display may be a modular system, or a separate components system, for example a computer, a separate monitor, and a separate camera. In another embodiment, the fitness manager interactive display may include a mobile computing device (e.g., tablet, mobile phone) configured with an appropriate camera. The fitness platform may be accessed using the mobile computing device.

At 210, the user logs in to the fitness platform (e.g., using fitness manager interactive display 204) to access their personal account and profile. In one embodiment, at 212, an initial physical assessment of the person may then be performed using the fitness manager interactive display. The physical assessment may include the fitness manager interactive display generating a portrayal of the person, including the portrayal being based on physical measurements identified for the person using an appropriate infrared or lidar camera. As previously discussed, the portrayal may be represented as a skeletal figure, a stick figure, a caricature representation, a bodily form representation, or any other feasible representation of the person. The portrayal allows the person to identify biomechanical feedback, physical movement, form, alignment, positioning, and/or stress of himself or herself during fitness training, as represented by the portrayal. The physical assessment, represented by the portrayal, is displayed on the fitness manager interactive display. The physical assessment takes into consideration any specific physical features, strengths, and/or weaknesses of the person prior to performing personal fitness training. The biomechanical feedback from the physical assessment, as displayed relative to the portrayal or other audio or visual cues associated with the fitness manager interactive display, helps to ensure safety and proper form during the fitness training.

In one example, this physical assessment is determined based on the user answering a few questions about their current health and historical injuries. For example, if a user mentions injuries that could affect their ability to do a particular assessment, that assessment will be avoided (e.g., someone who has recently had knee surgery would not be tasked with doing a parallel squat). The results of the assessments and history survey are used to determine aspects and elements of a workout, including for example, (1) range of motion for particular exercises, (2) exercises to begin with, (3) goal related information to track and display during the end of session summaries and between exercises, and (4) a potential progression plan for the user to pursue.

A user's profile and assessment information may be stored in the fitness manager interactive display 204, or stored directly to a network computing resource server or the cloud, or may be later uploaded to a network computing resource or the cloud. This network facilitation enables the user to access their personal profile from any computing platform, kiosk, mobile app, or other computing device the user may be using in coordination with the personal fitness training computing system.

Example user assessments that may be conducted prior to engaging in an exercise, to supplement data based on user reported or observed issues as recorded in the user's profile, may include:

1. Overhead squat (unloaded)

2. Standing assessment (facing camera)

3. Standing assessment (side view)

4. Single leg squat

5. Arm extension, rotation, raise and front lift

The overhead squat is a useful assessment as it is one that kinesiologists recognize as being able to help identify a variety of issues relating to mobility, including with respect to detecting issues relating to shoulder, chest, back, hamstring, etc. Similarly, simple standing assessments help determine the user's initial body form status.

By completing the initial questionnaire and assessments, users will have generated their base profile. Based on the profile, a user's initial exercise regimen and range of motion for exercises is determined and added to their profile. The user, or even a hired personal trainer or other representative, may have the ability to modify exercises at their discretion either via the fitness manager interactive display itself, or via a complementary mobile device, or via a computer or browser web portal.

At 214, the user selects a workout for the fitness training, such as from the initial personal profile creation, and if desired, at 216, may alter the workout parameters. At 218, the user's portrayal is displayed on the fitness manager interactive display, and the user performs their fitness training workout and may reference the portrayal and fitness manager interactive display for a real-time perspective of their fitness training. The fitness training is monitored, and performance data is captured by the fitness manager interactive display. Monitoring the fitness training and capturing performance data may occur with the fitness manager interactive display using the camera or a video camera associated with the fitness manager interactive display, and performance data may be saved to the fitness platform. For example, the data may be saved locally at the fitness manager interactive display first, then later uploaded to the cloud to be saved in the fitness platform, or the data may be saved directly to the fitness platform in real time, depending on design, networking, and data transfer options. As the fitness training workout is monitored, and performance data is captured, biomechanical feedback and other feedback indicators are provided relative to the fitness manager interactive display, including with respect to visualizing the portrayal and/or audio prompts.

Other visual or audio indications may also be provided as feedback. For example, the visual indication may be a video, an image, a color, a graphic, a line, a circle, a geometric figure, a symbol, or a written language indicator displayed relative to the portrayal or otherwise referenced with respect to the fitness training. The biomechanical indications of feedback may include identifying aspects related to a body joint, body form, body alignment, and/or body positioning, with respect to the physical activity, and may be represented by displaying a visual variation of the portrayal or other visual or audio indicators.

The fitness manager interactive display may also provide additional personal fitness training features. For example, such features may include a rest timer, a summary feedback and statistics at an end of a set of the physical activity, a summary feedback and statistics at an end of the entire physical activity, a virtual trainer, an adjustment of the physical activity relative to data collected from monitoring the physical activity, a social media feature, a gamification feature, a physical calibration, and/or a tutorial. The fitness manager interactive display may also be configured to recognize color or objects associated with the physical activity including, for example, different weight types or sizes, weight bars, weight plates, dumbbells, kettlebells, etc., to enable automatic load counting for a workout or for other information and feedback purposes.

In one embodiment, the fitness manager interactive display automatically adjusts user workout routines based on performance data captured. For example, by tracking various performance data such as form score, volume lifted, average velocity of movements, time under tension, tolerance, a range of motion, and other training related data, an individualized fitness plan may be developed and provided that helps progress goals or provides short digressions to help pass plateaus, avoid injury, and avoid overexerting certain muscle groups. By referencing form score and other observable data, the level of safety and comfort for a user while exercising is enhanced.

At 220, overview completed workout data may be viewed by the user, and recommendations for a next workout may be obtained. At 222, if the user has a personal trainer, then at 224 the trainer's coaching data may be referenced with respect to the trainer's computer at 206, and at 226 the trainer may assist in creating a new workout, which are then saved to the fitness platform for reference by the user's profile. On the other hand, at 222, if the user does not have a personal trainer, then at 228 the user may later review their detailed workout data on their own computer, browser, or mobile device at 202. The user may then plan their own next workout at 230, and save the data to the fitness platform.

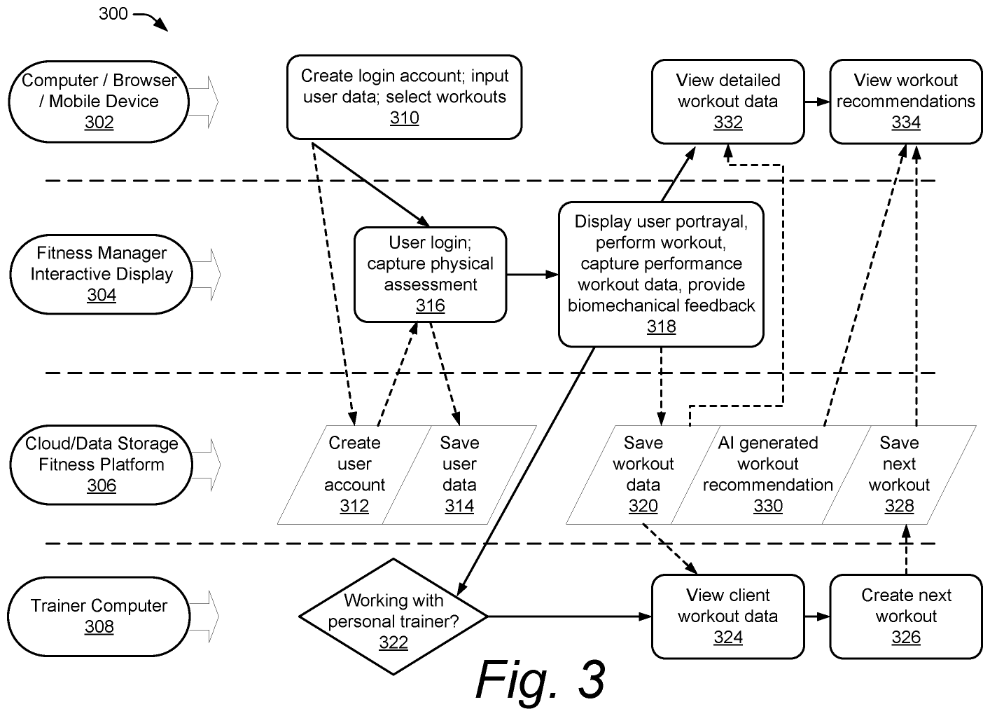

FIG. 3 illustrates an example overview of data flow associated with a personal fitness training computing system with biomechanical feedback. In this example, the computing system may include at 302 a computer, browser, or mobile device, at 304 a fitness manager interactive display device, at 306 a cloud/data storage fitness platform, and at 308 a fitness trainer computer. A user's flow of actions taken are indicated using solid lines with directional arrows. Data flow is indicated using dashed lines with directional arrows. At 310, a person (e.g., user) that intends to participate in a customized personal fitness training activity, interacts with the computer, browser, or mobile device at 302 to create a personal account, input personal data to create a personal profile, and select specific workouts or other activities for fitness training purposes. In an alternate embodiment, the person may create the personal account and input personal data directly into the fitness manager interactive display at 304. The personal account and personal data are created and saved 312, 314 on the cloud/data storage fitness platform at 306, for access by the computer, browser, or mobile device at 302, and fitness manager interactive display at 304.

When the person is ready to perform their fitness training, they login 316 at the fitness manager interactive display at 304, and access their personal profile and data stored on the fitness platform 306 at 312, 314. If an initial physical assessment of the person is performed, and/or a portrayal of the person is generated for an assessment, then that data is also saved 314 to the cloud/data storage fitness platform at 306.

At 318 the user's portrayal is displayed on the fitness manager interactive display, and the user performs their fitness training workout and may reference the portrayal and other visual and/or audio indicators for a real-time perspective of their fitness training. The fitness training is monitored, and workout/performance data is captured by the fitness manager interactive display and saved 320 to the cloud/data storage fitness platform at 306. As the fitness training workout is monitored, and performance data is captured, biomechanical feedback and other feedback is provided on the fitness manager interactive display 304, including with respect to visualizing the portrayal.

At 322, if the person is working with a personal trainer, then the personal trainer may view the person's (e.g., their client's) performance workout data at 324 as saved 320 on the cloud/data storage fitness platform at 306. The personal trainer may also create a next workout 326 for the client and save that data at 328 onto the cloud/data storage fitness platform at 306. In one embodiment, a next workout recommendation may also be automatically generated by the computing system, for example, using artificial intelligence principles and algorithms based on the person's performance data captured, and saved at 330 on the cloud/data storage fitness platform at 306.

At 302, on the computer, browser, or mobile device, the person may view their detailed workout data at 332 as obtained from the cloud/data storage fitness platform 306 at 320. At 334, the person may also view any computer system generated workout recommendation 330 and/or personal trainer next workout saved 328.

The system and/or operations of methods described in this disclosure may be embodied in whole or in part as programming instructions, for example, firmware or software. The instructions may be stored on a transitory or non-transitory, machine-readable medium (e.g., computer/processor-readable medium), including, for example, a random-access memory (RAM), read-only memory (ROM), flash memory, cache memory, solid-state drive (SSD), hard disk drive (HDD), or combinations thereof, for execution on one or more processors in one or more computing systems. In some examples, implementing the operations of these methods may be achieved by one or more processors, or processor cores, reading and executing the programming instructions stored in the memory. In other examples, implementing the operations of the methods may be achieved using an application-specific integrated circuit (ASIC), such as a system-on-chip (SOC), and/or other hardware components either alone or in combination with programming instructions executable by one or more processors in one or more computing systems.

The example methods described in this disclosure may include more than one implementation, and different implementations may not employ every operation presented in a respective flow diagram, or may employ additional steps not shown in the respective diagram. Therefore, while the operations of methods are presented in a particular order within the flow diagrams, the order of their presentations is not intended to be a limitation as to the order in which the operations may actually be implemented, or as to whether all of the operations may be implemented. For example, one implementation might be achieved through the performance of a number of initial operations, without performing one or more subsequent operations, while another implementation might be achieved through the performance of all of the operations.

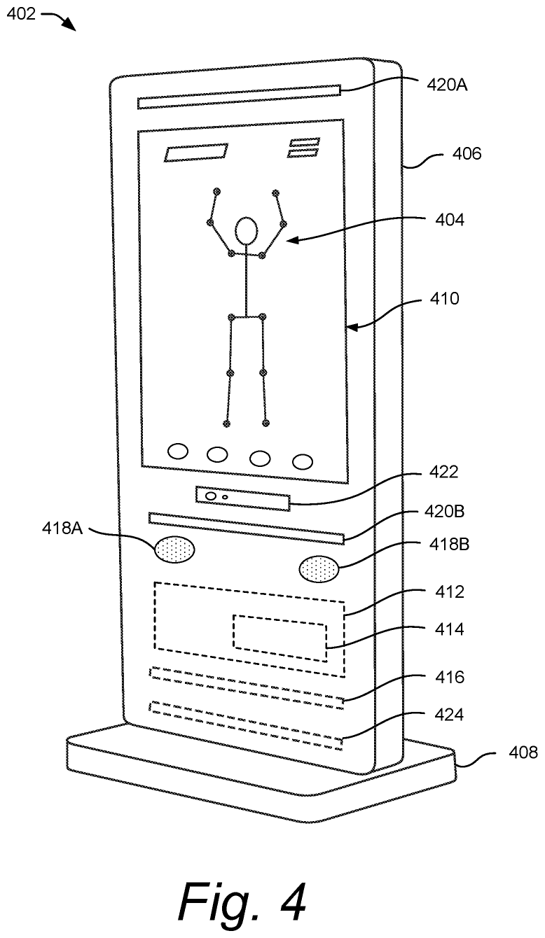

FIG. 4 illustrates an example personal fitness manager interactive display depicted as an all-in-one free-standing kiosk 402, serving as part of a personal fitness training system, and displaying an example biomechanical feedback in the form of a portrayal 404 of a person (e.g., user) performing fitness training. The actual person performing the fitness training, from which the portrayal 404 is generated, is not shown in the illustration. Only the portrayal 404 on the personal fitness manager 402 is shown in this example illustration.

In an alternate embodiment, the personal fitness manager interactive display 402 with biomechanical feedback may be configured physically differently, for example to hang on a wall or other support structure for use by the user, rather than be free-standing as shown. Other example configurations may include a smaller, portable unit to easily use at home or the office, or a more modular unit that allows integration with existing components, for example a television or computer monitor, infrared camera, and/or speakers. The modular option may allow a user to add in additional features, functionality, or components, such as light bars, screen size, a heart rate monitor system, a recording camera for integration into social media, additional motion or infrared cameras to allow multiple users to workout at a time, and modify the color scheme of the structure for aesthetics. Other design features may include being lighter in weight, and able to move components (e.g., a visual display or camera) at various angles. A truly mobile version may also be deployed through a handheld device, such as mobile phone or tablet device. Each of these configurations provides for broader and more mobile use while enabling biomechanical feedback for custom fitness training.

In this example shown, portrayal 404 is depicted as a skeletal (e.g., stick figure) representation of the person using the kiosk. For structural integrity and durability, this example kiosk may include a housing 406 composed of cold-rolled steel for durability and a steel base 408 for stability. This ensures that the kiosk can take potential hits (e.g., from weight bars, weight plates or people) so that the kiosk will incur minimal damage. Other structurally sound materials may also be used for the housing to ensure structural integrity of the kiosk. The kiosk may include an interactive display 410 (e.g., touchscreen), an internal computer 412, and a personal fitness manager 414 configured to operate with, and/or execute on, the computer to provide feedback, including biomechanical feedback, as described in this disclosure. The kiosk may also include an emergency power system 416 and speakers 418A and 418B. Some components of the kiosk may be configured inside the housing, or otherwise located relative to the housing for improved protection. The kiosk may also include light bars 420A and 420B, an infrared (IR) camera 422 (or lidar camera), and a wired or wireless network/Internet connection 424 to communicate with other networked computing devices such as a computer, browser, mobile device, and/or trainer computer as previously described.

The computer 412 may include standard computing resources such as a circuit board, a central processing unit (CPU), a graphics processing unit, memory, firmware, software, input/output functionality, and wired and/or wireless networking communication functionality. The computer may include other electronics, such as discrete electronic components and/or an application specific integrated circuit (ASIC). Memory may include volatile (e.g., RAM) and/or nonvolatile memory components (e.g., ROM, flash memory, etc.). The components of the memory comprise non-transitory, machine-readable (e.g., computer/processor-readable) media that can provide for the storage of machine-readable coded program instructions, data structures, program instruction modules, and other data and/or instructions executable by a processor. Thus, the computer generally comprises a processor programmed with instructions, or capable of receiving instructions or data, such as from a mobile device or network resource, that when executed, cause the computer and kiosk to function and interact with a user as described herein.

The IR camera 422 captures a dot map image of a person using the kiosk 402. The personal fitness manager 414 references the dot map image to generate the portrayal 404 of the person to provide feedback relative to physical activity associated with the personal fitness training being performed. Joints and movement associated with the physical activity of the person are highlighted or otherwise reflected on the portrayal 404. The portrayal allows the person doing the fitness training to identify biomechanical feedback in real time, including physical movement, form, alignment, positioning, and/or stress during fitness training.

The interactive display 410 enables a person to engage with settings provided by the personal fitness manager 414 and to access functionalities of the kiosk 402. The personal fitness manager 414, in coordination with the interactive display 410 and speakers 418, provide feedback relative to the person's physical activity, either of a visual or audio indication. For example, a visual indication may include the portrayal 404, a video, an image, a color, a graphic, a line, a circle, a geometric figure, a symbol, or a written language indicator, either directly associated with or separate from the portrayal 404. The biomechanical feedback may include an indication identifying factors including a body joint, form, alignment, or positioning, and/or stress with respect to the person's physical activity as represented with respect to the portrayal 404.

The light bars 420A, 420B may enhance the accuracy of the infrared camera by ensuring the person is well lit to create an accurate dot map and portrayal (e.g., from a front profile or any other perspective). The light bars 420A, 420B, IR camera 422, and personal fitness manager 414, also enable color and object recognition, for example, to recognize different weight types or sizes, weight bars, weight plates, dumbbells, kettlebells, etc., to enable automatic load counting for a workout or for other information and relevant feedback purposes.

The personal fitness manager 414 may also automatically adjust user workout routines based on performance data collected during real-time tracking (e.g., using the IR camera 422) of the user during an exercise workout. By tracking various performance data such as form score, volume lifted, average velocity of movements of muscle areas worked, time under tension, and other training related data, the personal fitness manager is able to develop individualized plans that help progress goals or provide short digressions to help pass plateaus, avoid injury, and avoid overexerting certain muscle groups.

By incorporating form score to other observable data, the personal fitness manager 414 may guide the user through ways to enhance their mobility or range of motion. This may increase safety and comfort for the user while they exercise, and unlock future exercises that maybe they shouldn't have been doing before for safety and comfort without that mobility or range of motion. For instance, even if a user is able to safely complete a certain lift (e.g., goblet squats) but the form score is lower than the average of a user's other lifts by upwards of a predefined threshold (e.g., 5%), the personal fitness manager will flag this exercise as in need of a possible replacement. If the discrepancy continues through future exercises, the personal fitness manager 414 encourages the user to make the switch to an exercise that works the same muscle groups but has a higher likelihood of a better form score.

A similar event would encourage a user to start a digression series in a workout. For example, if a user has a strength goal, and after 4 weeks of lifting the system notices a drop in form score but consistent velocity training for a particular lift (e.g., deadlift), then the personal fitness manager 414 will flag the lift. Then, during the next two times (or other designated times) the user does deadlifts, if the form score drops lower and velocity falls from targeted speeds, then the personal fitness manager 414 will encourage the user to begin a digression series to avoid injury and to eventually return to the current load levels.

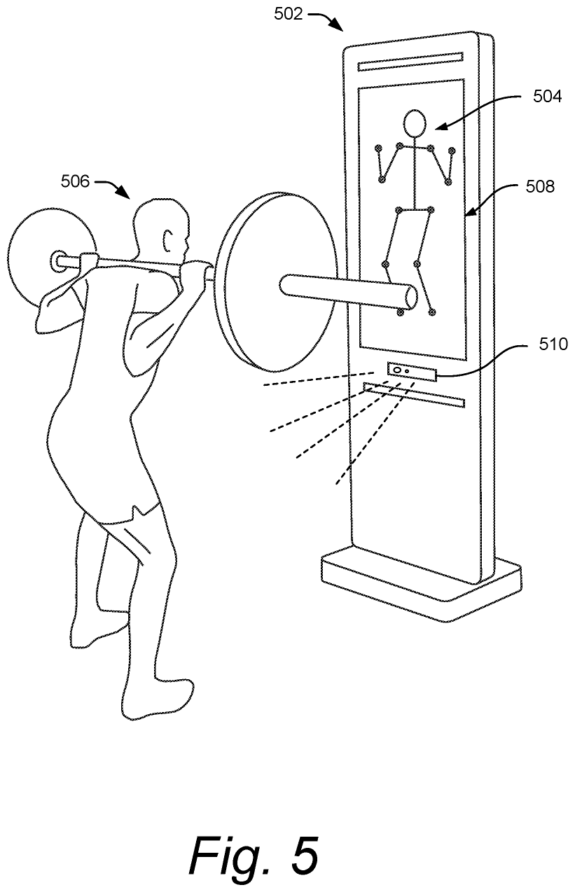

FIG. 5 illustrates an example personal fitness manager interactive display 502 depicted as a free-standing kiosk, with biomechanical feedback displayed in the form of a portrayal 504 of a person (e.g., user) 506 performing fitness training. The fitness manager interactive display 502 is placed in front of, and is being used by, the user 506 performing a workout. In this example, the fitness manager interactive display is a free-standing kiosk, but in alternate embodiments may be configured to hang on a wall or other support structure for use by the user, or may be configured in other modular or mobile properties. Also in this example, the user is lifting weights for their workout while interacting with the fitness manager interactive display 502 and receiving instructions and guidance from the fitness manager interactive display, including the biomechanical visual feedback depicted by the portrayal 504 on the interactive screen 508. However, other fitness training activity may similarly be performed by the user 506, and the personal fitness manager interactive display 502 provides feedback relative to the fitness training.

In one example, the portrayal 504 of the person 506 is generated by first having the infrared camera 510 capture a dot map image of the person 506. Then the personal fitness manager interactive display 502 creates the portrayal 504 from the dot map image, displays it on the interactive screen 508, and monitors the person doing the fitness training to provide real time feedback on the interactive screen 508 or via an audio function associated with the personal fitness manager interactive display 502. The feedback may include biomechanical feedback (e.g., the portrayal 504), as well as other visual and/or audio feedback relating to the fitness training activity.

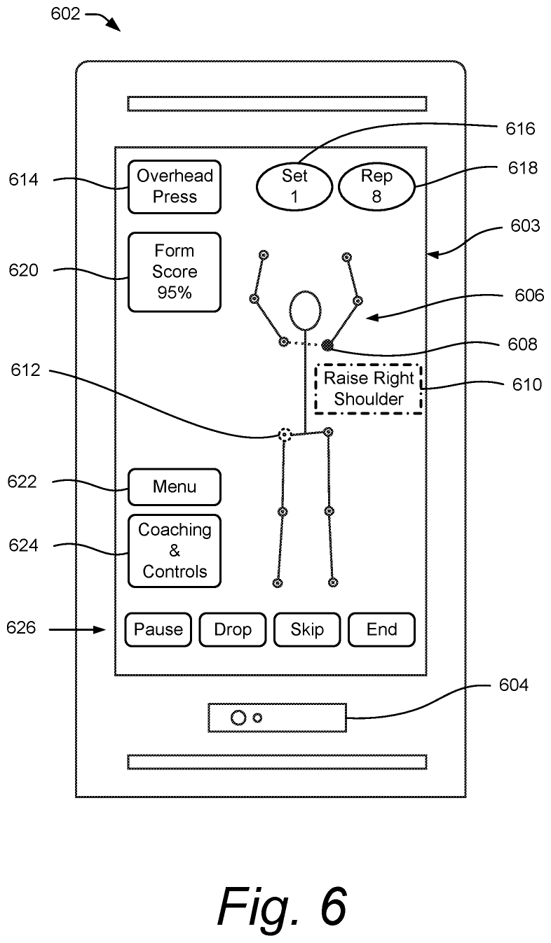

FIG. 6 illustrates example user interface features and functionality of a personal fitness manager interactive display 602, including providing biomechanical feedback on touchscreen 603. In this example, an infrared camera 604 previously captured a dot map image of a user, and the personal fitness manager interactive display 602 used the dot map image to generate the portrayal of the skeletal figure (e.g., stick figure 606 built to represent the user. As the interactive display monitors the user's physical activity in real time with the infrared camera 604 or other video camera, the user's physical motion and joints are reflected on the touchscreen 603 real-time in the skeletal portrayal 606 to depict how the user is moving their body and joints. A correction algorithm is run over their body at each frame of the camera capture (e.g., 30 frames per second) to analyze body positioning continuously and in real-time. Using the skeletal points and scientifically referenced best movement patterns (e.g., kinesiology studies), the fitness manager interactive display 602 provides feedback to the user, in the form of the portrayal, visual images, video, text, and/or audio comments, describing how to correct their positioning and form to do the current exercise correctly.

Visual feedback may be provided as various visual cues relative to the exercise being performed. For example, the skeletal portrayal 606 may be visually represented using different colors, highlights, symbols or other indicators to identify different aspects of movement, stress, or form error. Visual messages may also be displayed. As an example, on an initially grey or white colored portrayal, a user will see joints specifically identified as primary errors that are moving most incorrectly, unsafely, or out of alignment (e.g., outside of best patterns). Those areas will be highlighted with an identified color (e.g., red) 608 (depicted as a bold circular shoulder joint at 608) during the exercise if extremely out of form or unsafe. In one embodiment, the personal fitness manager interactive display 602 automatically adjusts guidance and feedback based on biomechanical adaptations of the individual user. For example, if a user's particular physical measurement is identified outside of norms, say a particular hip to femur alignment, the personal fitness manager will adjust to have the individual track a different way (e.g., outside of normal best patterns) when doing squats as compared to most people. This automatic adjustment will be best for the particular user, but not for other people. So tolerances may be adjusted based on a particular user's need. Optionally, additional written feedback, for example “raise right shoulder” 610, may be displayed to inform the user how to correct the primary error. An audio indicator may also inform the user of the error or how to correct it.

A different color or highlight indicator may inform the user of a secondary error occurring. This may represent an error that is not the most concerning at the time, but relevant or important to note. Once the primary error is corrected, the secondary error may re-color to the primary error color. For example, another color (e.g., yellow) 612 may be used to highlight areas if somewhat out of form or unsafe (depicted as a dashed circular hip joint at 612). Similarly, upon full contraction or extension (concentric portion of a movement) the entire skeletal image may turn another color (e.g., green) indicating a correct final position. Alternatively, another color (e.g., yellow) may be used to highlight areas if a few parts of the body are out of alignment from accepted tolerances, or yet another color (e.g., red) if a substantial number of body parts are out of alignment or a particular movement area is extremely poorly positioned.

Additional example feedback may include a written indication of the exercise being performed, for example overhead press 614, a set number 616, and a repetition counter 618. Each of these may provide interactive functionality. For example, the repetition counter may display repetitions completed by the user and may be adjusted at the start of an exercise. Upon completing the set of repetitions, or by touching an indicator associated with the interactive repetition counter to decrease the repetitions, the set may end.

Another visual cue may involve a written prompt appearing on the touchscreen 603 indicative of a form score 620 for the exercise being performed. This form score displays a measure of efficiency for the user with respect to the exercise being performed and is also indicative of a safety feature relative to overall body form during the workout, or relative to the worst error of body positioning. For example, it may be on a scale of 0% to 100%, and may be based on the number of errors committed by the user compared to possible errors that could have been committed. It may be indicative of a qualitative or quantitative assessment of correctness, conformity, stress, potential injury, or any other similar performance evaluation. It may quantify movement conformity to a reference movement profile. It may also be based on a severity multiplier relative to the error committed. For example, a detected spinal twist or tilt may be recognized as more severe than a slight foot misalignment, so a spinal error will reduce form score more than a foot misalignment. Form score may be used to encourage correct form and to adjust the workout as necessary. For example, if multiple errors are being committed, the most misaligned or “at-risk of potential injury” area of the body may be commented on with the visual form score or audible prompt. The form score prompt may provide an additional cue of how to safely correct the form issue.

A menu button 622 may allow a user to effectuate different options relative to the personal fitness manager interactive display 602, including for example, select an exercise (e.g., overhead press workout 614), edit an exercise, manage a user profile, participate in social media features or gamification features, adjust exercise calibration features, participate in tutorials, and/or select premium purchase options. A user or trainer may touch the touchscreen 603 interface of the fitness manager interactive display 602 for prompts to appear for the workout to be changed. A user may change the workout before or during the workout, and may select the workout they wish to change and select specific exercises. A user may also change factors such as sets, reps, load or exercise, or the workout of a particular day. A user may also change exercise routines as a whole by adding workout history or selecting future progressions they seek to follow. These and similar functionalities and options are likewise available through a mobile device (e.g., mobile application) or by login to the fitness platform through a network connected computer or browser.

The fitness manager interactive display 602 may also include a coaching and controls feature 624. By selecting this option, a user may see written descriptions, or watch images or videos, describing or teaching about a specific exercise, proper form, and/or other fitness considerations. Additional interactive control features 626 may allow the user to pause, drop, skip, or end a specific workout.

Other interactive feedback (not shown) may include a rest timer, and/or a weight load, indicating current weight being lifted as detected by the personal fitness manager interactive display and camera. Additional feedback may include a safety visual prompt in the case of too many errors continually being committed under load. This prompt may be in the form of indicators including, for example, a graphic image, written wording, or audio announcement, and informs the user to return to starting position and place the weights in the starting position (e.g., rack the weights in holders, place weights on the floor, etc.). Upon returning the weights, the fitness manager interactive display will walk the user through the movement without load to ensure the portrayal (e.g., dot map generated skeleton) is properly calibrated. If the calibration goes well, the user is prompted to continue the movement again. If the calibration shows there are errors in the person's movement without load, the user may be shown a training video where they see a coach doing the movement, and learn what biomechanical cues are important for the movement being performed. The user may then be visually prompted to mirror the on-screen coach's movements with visual feedback again being provided. This process is to ensure that the user can adapt to the movement requirements. If movements do not improve from the calibration phase, the workout may move to another exercise.

An interactive rest timer may also be displayed between each set indicating how much time they have to rest between sets. The user may increase or decrease this amount based on needs. Based on scientific guidelines and users goals, there is a suggested rest time provided for each workout. At the end of each set, in conjunction with the rest timer, a user may be given visual and/or audio feedback on how to improve the exercise for a future set. This feedback points out the most common error committed and how to improve it. In some cases, the fitness manager interactive display may suggest changing the exercise or add a new exercise to address this error. The feedback also provides statistics about the exercise performed. This may include average velocity, velocity by rep, time under tension, volume lifted, the average range of motion, range of motion per rep, time of set, current workout time and/or additional data that would be beneficial to a user.

If at any time the user doesn't know how to do an exercise, they may touch the input interface (e.g., touchscreen 603) for information or instructions (e.g., menu button 622). Upon doing so several prompts will appear (e.g., skip exercise, end workout, add exercise, add set, show me how to do this). If a user selects a message such as “Show me how to do this” then a virtual trainer may appear on the screen (e.g., pre-recorded). The trainer will show the movements, and the screen will show what the key components to the movement are.

Alternatively, or in addition to any visual or written feedback indicator, audio feedback may be provided through sources such as built-in speakers on the fitness manager interactive display, attachable speakers, or Bluetooth headphones that connect to the fitness manager interactive display through the user's mobile device (e.g., cell phone) where a complementary mobile application is installed. Similar to visual feedback, audio feedback provides cues to help correct errors a user may be making, among other things. Audio feedback also focuses on the primary error and encourages that to be corrected. Additionally, during a user's rest in between exercises, the audio feedback may provide encouragement and provide prompts for how to do or how to improve the workout during the next exercise. Moreover, visual, textual, and audio indicators may be used to congratulate, encourage or motivate a user. For example, a user may be welcomed on their birthday, their xth workout anniversary, gamification successes, social media comparisons, and the like.

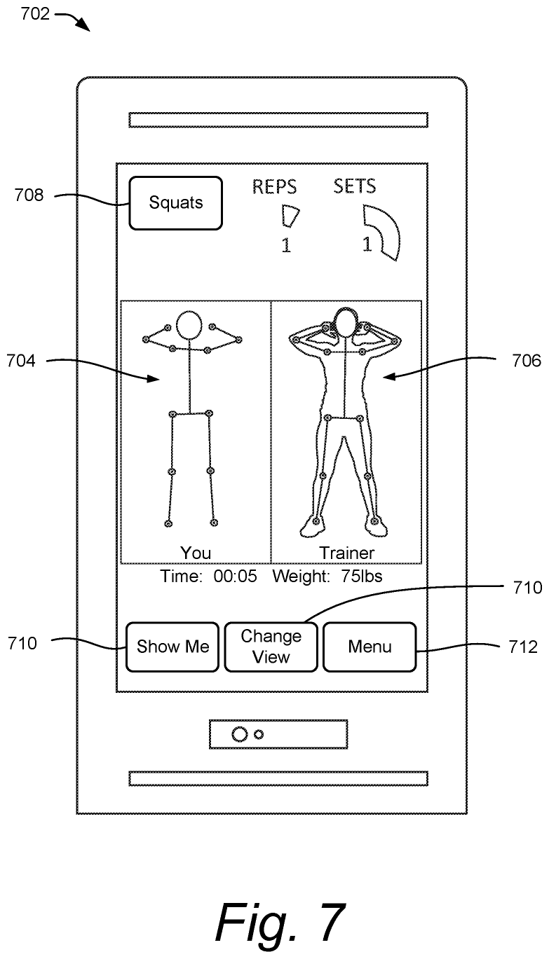

FIG. 7 depicts a personal fitness manager interactive display 702 user-interface illustrating an example of the user's skeletal portrayal 704 positioned side-by-side with a virtual trainer's portrayal 706. This option and feedback provides a biomechanical comparison of body form for the specific exercise, such as for a squat exercise 708 depicted in this example. However, this feature may be provided for any exercise (e.g., dead lift, overhead press, lunge, curl, or using bar, dumbbell, kettlebell). If at any time the user doesn't know how to do an exercise, or simply desires further guidance, they may select the appropriate option, for example, “show me” button 708, “change view” button 710, or “menu” button 712, for information or instructions. A virtual trainer 706 will appear on the screen to show the movements. This information may also be expressed via audio or written text.

Once the cues have been shown, the user will be prompted to mimic the trainer without any load/resistance. If the user has a form score greater than an acceptable threshold (e.g., 90%) on a given number of consecutive reps (e.g., three), then the user may continue on to add load. If the form score stays below the threshold for more than a given number of reps (e.g., five) then the user will see the tutorial portion by the trainer again. Only the cues they are struggling with will be shown during this repeat. However, once the specific cues are shown, the personal fitness manager interactive display 702 shows common ways to fix problems preventing the correct movement. The user is then guided to mimic the movements again, after which the user can elect to move on to the load portion of the workout or move on to another exercise.

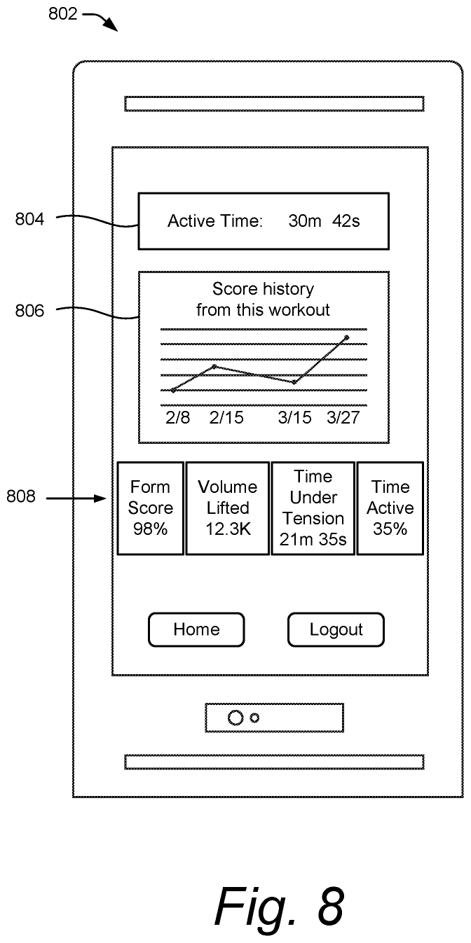

FIG. 8 illustrates an example personal fitness manager interactive display 802 depicting user-interface features of a workout summary after a customized personal fitness training workout is completed. Upon completing all of the parts of a workout a user may receive feedback on the entirety of the workout. This may highlight selected factors or statistics of the workout. For example, aspects displayed may include active time 804, score history 806, and/or other relevant data 808 such as form score, volume lifted, time under tension, and time active. Other summary data may include the most improved exercise or the most recurring challenge area of the body. Suggestions may also be displayed for how to improve upon the challenged error in future workouts. Future workout plans may also be modified if an error was extreme across several exercises. Additional statistics may include average velocity, velocity by rep, average range of motion, range of motion per rep, time of set, current workout time and/or additional data that would be beneficial to the user. Any of this data may also be accessed via a mobile device or through a computer or browser login to the fitness platform.

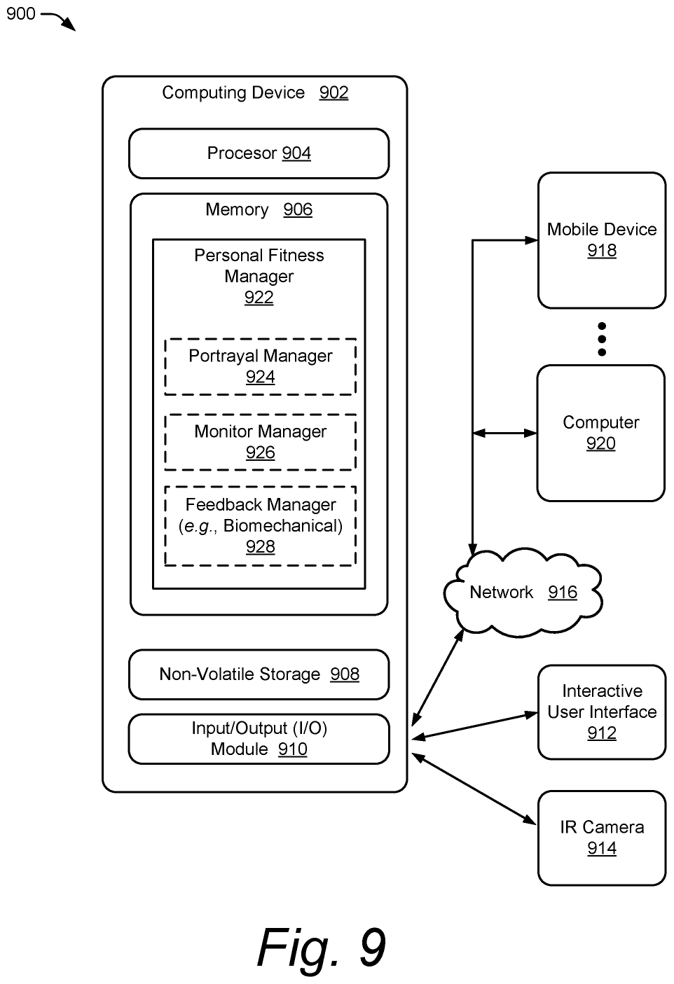

FIG. 9 is a block diagram 900 illustrating an example computing device having a personal fitness manager and interactive display. The computing device 902 may include a processor 904, a memory 906, a non-volatile storage 908, and an input/output (I/O) module 910. The computing device communicates with an interactive user interface 912, an infrared camera 914, and a network 916 through the I/O module 910. The user interface 912 and camera 914 may be incorporated as part of the computing device 902, or implemented separate from the computing device. As some non-limiting examples, the computing device 902 may include components commonly associated with a computer server, desktop computer, notebook computer, mobile device, handheld device, or other computing device or system. The processor 904 may be any central processing unit (CPU) with one or more cores. The memory 906 may be any transitory and/or non-transitory, machine-readable media (e.g., computer/processor-readable media), such as a RAM, ROM, flash, cache, SSD, or a combination thereof, for storing program files, data, an operating system, and other executable instructions. The non-volatile storage 908 may be any ROM, flash, HDD, SSD, or similar storage device, for permanently storing program files, data, an operating system, and other executable instructions.

The interactive user interface 912 may be a display, monitor, screen, touch screen, keyboard, mouse, other input or output device, or any combination configuration of these devices, to display or otherwise provide information from the computing device to a user, and to allow the user to input information into the computing device. The camera 914 may be an infrared or lidar camera, or similar camera with video functionality, capable of capturing a dot map image. The network 916 may be any local area network (LAN), wide area network (WAN), cloud, multi-cloud, hybrid cloud, or private on-premise cloud computing environment, by which the computing device 902 may communicate with other networked devices, computing devices, or systems. In this example, the network 916 allows the computing device 902 to communicate with other network connected devices, including mobile device 918 or other computer 920.

Memory 906 includes a personal fitness manager 922 configured to operate with the computing device 902 to, among other things, generate a portrayal of a person performing fitness training, monitor their fitness training, and provide feedback, including biomechanical feedback, according to principles and methods described in this disclosure. In this example, the personal fitness manager 922 is depicted simply as an application configured with executable instructions, for instance, firmware or software, for execution on the processor 904. However, the operations of the personal fitness manager 922 may be implemented in whole or in part using an ASIC, such as a SoC, and/or other hardware components either alone or in combination with programming instructions executable by the processor.

The personal fitness manager 922 may include manager modules to implement its functionality, for example, a portrayal manager 924, a monitor manager 926, and a feedback manager 928. These manager modules are shown separately in the illustration for discussion purposes, but may be combined, or further separated into additional modules, according to design and functional preference. Each of these manager modules functions according to methods and principles described in this disclosure for enabling a personal fitness training system with biomechanical feedback. For example, the portrayal manager 924 may function as previously described with respect to coordinating with the infrared camera 914 to capture a dot map image of a user, generate a portrayal of the user based on the dot map image, and display the portrayal on the interactive user interface 912. The monitor manager 926 may function as previously described with respect to monitoring a physical activity of the person during their personal fitness training. Similarly, the feedback manager 928 may function as previously described with respect to providing feedback to the user relative to the physical activity, including biomechanical feedback, by displaying visual feedback on the interactive user interface 912 or providing audio feedback through a sound option associated with the computing device 902 and/or input/output module 910.

Although the personal fitness manager 922 is shown as an application with executable instructions in the memory 906, this is simply illustrative for discussion purposes. For example, the personal fitness manager 922 may be executed locally on computing device 902 as shown, or executed remotely via network 916 relative to a separately networked computing device, such as using a software as a service (SaaS) model, or a combination of local and remote execution.

While this disclosure has been described with respect to example embodiments outlined above, it is evident that alternatives, modifications and variations will be apparent to those skilled in the art. Accordingly, the described and depicted embodiments of the present disclosure are intended to be illustrative, not limiting, and the subject of the appended claims is not necessarily limited to the specific features or methods described herein.

What is claimed is:

1. A method of customizing personal fitness training, the method comprising:
generating, by a computing device, a portrayal of a person, the person identified for participating in the personal fitness training;
displaying the portrayal on a display device communicating with the computing device;
monitoring a physical activity of the person, the physical activity associated with the personal fitness training; and
providing, on the display device, feedback relative to the physical activity, the feedback comprising a biomechanical indication of the physical activity represented with respect to the portrayal.

2. The method of claim 1 wherein generating the portrayal comprises:
generating a dot map representation of a physical assessment identified for the person; and
creating the portrayal from the dot map representation.

3. The method of claim 1 wherein the portrayal comprises at least one of a skeletal figure, a stick figure, a caricature representation, or a bodily form representation.

4. The method of claim 1 further comprising the computing device receiving data indicative of a personal profile of the person, and guiding the person through the physical activity relative to the personal profile, to identify a physical assessment of the person for generating the portrayal.

5. The method of claim 1 further comprising receiving, by the computing device, input data indicative of the physical activity to be performed.

6. The method of claim 1 wherein the feedback further comprises at least one of a visual indication or an audio indication.

7. The method of claim 6 wherein the visual indication comprises at least one of a video, an image, a color, a graphic, a line, a circle, a geometric figure, a symbol, or a written language indicator.

8. The method of claim 1 wherein the biomechanical indication comprises an indication identifying at least one of a body joint, form, alignment, or positioning, with respect to the physical activity as represented with respect to the portrayal.

9. The method of claim 1 wherein the biomechanical indication of the physical activity represented with respect to the portrayal comprises a visual variation of the portrayal.

10. The method of claim 1 further comprising providing, by the computing device, information relating to at least one of: an error identified relative to the physical activity as conveyed by the feedback comprising the biomechanical indication of the physical activity, or how to improve the physical activity as conveyed relative to the feedback comprising the biomechanical indication.

11. The method of claim 1 further comprising providing, by the computing device, personal fitness training features relative to the physical activity comprising at least one of a rest timer, a summary feedback and statistics at an end of a set of the physical activity, a summary feedback and statistics at an end of the entire physical activity, a virtual trainer, an adjustment of the physical activity relative to data collected from monitoring the physical activity, a social media feature, a gamification feature, a physical calibration, or a tutorial.

12. The method of claim 1 further comprising recognizing, by the computing device, an object associated with the physical activity, and wherein the feedback provided is relative to the object.

13. The method of claim 1 further comprising capturing, by the computing device, performance data associated with the physical activity, and adjusting the physical activity for the person responsive to the performance data.

14. The method of claim 13 wherein the performance data comprises at least one of a form score, a volume lifted, average velocity of movement of muscle areas worked, a time under tension, a tolerance, or a range of motion.

15. The method of claim 13 wherein adjusting the physical activity functions to accomplish at least one of progressing in a goal, improving potential, providing a short digression, helping pass a plateau, avoiding an injury, avoiding overexerting certain muscle groups, enhancing a level of safety, enhancing a level of mobility, enhancing a range of motion, or enhancing a level of comfort for the person.

16. A personal fitness training system comprising instructions that, when executed by a computing device, cause the computing device to:
generate a portrayal of a person, the person identified for participating in the personal fitness training;
display the portrayal on a display device communicating with the computing device;
monitor a physical activity of the person, the physical activity associated with the personal fitness training; and
provide, on the display device, feedback relative to the physical activity, the feedback comprising a biomechanical indication of the physical activity represented with respect to the portrayal.

17. The personal fitness training system of claim 16 further comprising instructions that, when executed by the computing device, cause the computing device to:
generate a dot map representation of a physical assessment identified for the person; and
create the portrayal from the dot map representation.

18. The personal fitness training system of claim 1 wherein the portrayal comprises at least one of a skeletal figure, a stick figure, a caricature representation, or a bodily form representation.

19. The personal fitness training system of claim 1 wherein the biomechanical indication comprises an indication identifying at least one of a body joint, form, alignment, or positioning, with respect to the physical activity as represented with respect to the portrayal.

20. A non-transitory machine-readable storage medium storing instructions that, when executed by at least one computing device, cause the at least one computing device to:
generate a portrayal of a person, the person identified for participating in the personal fitness training;
display the portrayal on a display device communicating with the computing device;
monitor a physical activity of the person, the physical activity associated with the personal fitness training; and
provide, on the display device, feedback relative to the physical activity, the feedback comprising a biomechanical indication of the physical activity represented with respect to the portrayal.
